(19) United States
(12) Patent Application Publication
(10) Pub. No.: US 2003/0161842 A1
(43) Pub. Date: Aug. 28, 2003

(54) Pleurotus extract and use in treating hypertension

(76) Inventors: Rui Wang, Saskatoon (CA); Yaoge Huang, Saskatoon (CA); Weimin Zhao, Nepean (CA)

Correspondence Address: GREENLEE WINNER AND SULLIVAN P C, 5370 MANHATTAN CIRCLE, SUITE 201, BOULDER, CO, 80303, US

(21) Appl. No.: 10/327,642

(22) Filed: Dec. 20, 2002

Related U.S. Application Data
(60) Provisional application No. 60/341,209, filed on Dec. 20, 2001.

(51) Int. Cl. A61K 35/84

(52) U.S. Cl. 424/195.15

(57) ABSTRACT

The invention provides an extract of the Pleurotus genus such as Pleurotus eryngii (DC. et Fr) Quél to prevent and treat hypertension, and enhance cardiovascular health. Further, the invention provides pharmaceutical compositions containing the extract as an active ingredient, in a pharmaceutically or therapeutically acceptable carrier. The invention also provides a method of preventing and treating hypertension by administration of the extract. Further, there is provided a method for preparing the extract and evaluating the extract using in vitro or in vivo tests to ensure antihypertensive activity.

### CROSS-REFERENCE TO RELATED APPLICATION

This application claims priority from U.S. Provisional Patent Application No. 60/341,209 filed Dec. 20, 2001, which is incorporated by reference herein to the extent that there is no inconsistency with the present disclosure.

### FIELD OF THE INVENTION

The present invention relates to a mushroom extract prepared from the genus Pleurotus such as Pleurotus eryngii (DC. et Fr.) Quél. and its preparation and use in the prevention and treatment of hypertension, and enhancement of cardiovascular health.

### BACKGROUND OF THE INVENTION

Hypertension is a disorder characterized by persistently high arterial blood pressure, whereby the systolic blood pressure (representing the pressure generated when the heart beats) remains consistently higher than 140 mm Hg, or diastolic blood pressure (representing the pressure in the vessels when the heart is at rest) remains consistently over 90 mm Hg. Hypertension can lead to stroke, heart attack or failure, cardiac arrhythmia, arteriosclerosis, or renal failure. Hypertension may have no known cause (“primary hypertension” or “essential hypertension”) or be associated with other primary diseases (“secondary hypertension”).

Comprising over 95% of all hypertension, “primary hypertension” or “essential hypertension” is of unknown etiology, but may have genetic or environmental origins (e.g., obesity, dietary sodium). Primary hypertension is asymptomatic until complications develop in target organs, and diagnosis depends on repeatedly monitoring the patient for persistently higher than normal systolic and/or diastolic blood pressure. While there is no cure for primary hypertension, treatment involves reduction of high blood pressure through lifestyle changes (e.g., weight loss, exercise, diet), and antihypertensive drugs including diuretics, beta-blockers, alpha-beta blockers, calcium channel blockers, alpha₁-adrenergic blockers, angiotensin-converting enzyme (ACE) inhibitors and angiotensin II receptor blockers. Although treatment is generally initiated with one drug, it is common for treatment to proceed with three or four drugs in combination (e.g., a diuretic with a beta-blocker or an ACE inhibitor). However, such drugs may be unsuitable for patients with particular conditions and tend to have deleterious side effects, such that the patient may have to try a number of different drugs in varying dosages before an effective and well tolerated treatment is identified. The antihypertensive drugs which are currently available present a range of side effects; for example, diuretics can precipitate diabetes or gout. Beta-blockers have adverse effects on the central nervous system (e.g., sleep disturbances, fatigue) and metabolism (e.g., increased serum cholesterol levels, glucose intolerance, decreased high density lipoprotein cholesterol), and can induce asthma, heart failure, or sexual dysfunction in men. Calcium channel blockers are potent vasodilators which do not induce adverse metabolic effects like beta-blockers; yet, particular calcium channel blockers (e.g., nifedipine) can cause edema and tachycardia, and are expensive. Adrenergic inhibitors have adverse effects on the central nervous system, inducing lethargy and depression. ACE inhibitors and angiotension II receptor blockers are preferred for treatment due to fewer side effects, but are highly expensive.

For severe hypertension, potent drugs (e.g., minoxidil, hydralazine, diazoxide, sodium nitroprusside) are administered. Minoxidil and hydralazine are potent, but have adverse side effects. Diazoxide and sodium nitroprusside are both administered rapidly by intravenous injection with adverse side effects including nausea, hyperglycemia and tachycardia (with use of diazoxide) and nausea, agitation, and muscular twitching (with use of sodium nitroprusside).

“Secondary hypertension” is due to or associated with a variety of primary diseases such as renal disorders (e.g., polycystic renal disease, kidney inflammation), disorders of the central nervous system, endocrine diseases (e.g., adrenal gland tumors, Cushing's syndrome, hyperparathyroidism), and vascular diseases. Unlike primary hypertension, treatment of secondary hypertension depends upon an identifiable cause which can be addressed accordingly; for example, when secondary hypertension results from a tumor or a blood vessel abnormality, surgery may be recommended. Antihypertensive medications may also be administered. High blood pressure can thus be cured when the underlying disease is treated successfully.

In the United States, hypertension affects nearly 50 million people, particularly African Americans. In Canada, approximately 22% of adults between 18 to 70 years of age are affected. Further, the disorder appears more prevalent in older individuals. As set out above, primary hypertension comprises 95% of all hypertension, with treatment encompassing lifestyle changes and drugs which have severe side effects. Since a combination of three or four drugs is often required, treatment is considerably expensive. Undesirable side effects and high costs contribute to an increased frequency of non-compliance by patients who are faced with lifetime medication. Empirical studies have demonstrated that in elderly individuals with systolic hypertension, 28 to 35% of patients did not reach their target blood pressure, 21% required medication other than a diuretic and a beta-blocker, and 13% ceased treatment due to adverse side effects. Since the drugs presently available have severe side effects and inadequate dosing regimes, there is a need for an alternative antihypertensive treatment which is highly effective, specific in its action, and minimal in its side effects.

The pharmaceutical industry has focused predominantly upon lengthy and costly development and manufacture of synthetic drugs; yet, several synthetic drugs have originated from natural, botanical sources. Western physicians have been reluctant to prescribe herbal medicines due to lack of scientific research of their preventative and therapeutic properties. However, herbal medicines are advantageous since they do not require the lengthy development time and high costs normally encountered with synthetic drugs. Further, they are readily available and offer the patient a more comfortable and affordable alternative with minimal side effects compared to prescription medication.

Various species of mushrooms reportedly are effective against hypertension and other diseases. Japanese Patent Application No. 01-256515 to Masaru et al. and Japanese Patent Application No. 2000-156548 to Satoshi et al. relate to isolation of antihypertensive components of Grifola frondosa (maitake mushrooms). Japanese Patent Application No. 10-323664 to Kazuhide relates to a composition containing G. frondosa and two other herbs for treating hypertension, diabetes, and liver function, while Japanese Patent Application No. 07-120675 to Chihiro relates to a single protein obtained from G. frondosa to treat hypertension, hyperlipemia, and obesity. Japanese Patent Application No. 57-216350 to Shigeru et al., Japanese Patent Application No. 55-187752 to Takeshi et al and U.S. Pat. No. 6,468,542 to Liu et al. relate to isolation of components of G. frondosa to treat hypertension, hyperlipemia, arteriosclerosis, thrombosis, immunological diseases, cancer, hepatitis, diabetes, AIDS and other diseases. Further, Japanese Patent Application No. 61-109156 to Akio et al. relates to a composition containing dried flowers of brown algae and shiitake mushrooms for sustaining normal blood pressure. The disadvantage of many such compositions is that several herbs are required as starting materials, or the composition is not specific for hypertension but a myriad of diseases including hypertension.

Various species of the genus Pleurotus have been reported to contain antihypertensive compounds. Japanese Patent Application No. 05-270240 to Shoichi et al. relates to a substance containing a fruit body or mycelium of Pleurotus sajor-caju for use as an antihypertensive. Japanese Patent Application No. 2000-377553 to Fumiharu et al. describes a Pleurotus eryngii strain obtained by a cell fusion of a protoplast prepared from two strains of eryngii obtained from an auxotrophic mutant, and a hypertension treatment agent of which the Pleurotus eryngii strain is the main component. The strain can be made into a powder which is then extracted in hot water to provide an anti-hypertensive composition. However, this method involving genetic engineering and several starting materials appears lengthy and costly. A natural, herbal composition which specifically targets hypertension without deleterious side effects, and can be readily prepared to minimize manufacturing costs is thus much desired.

### SUMMARY OF THE INVENTION

The present invention provides a novel extract of Pleurotus obtained by solvent extraction from the whole mushroom of the genus Pleurotus. A preferred mushroom source of the extract is Pleurotus eryngii (DC. et Fr.) Quél. The extract is preferably prepared from the whole mushroom, with the most preferred solvent including ethyl acetate.

The invention also provides a pharmaceutical composition comprising an extract of Pleurotus as set out above, in admixture with one or more pharmaceutically acceptable carriers.

The invention also provides a method of preventing and treating hypertension by administering a therapeutically effective amount of an extract of Pleurotus as set forth above to a host animal.

The invention also extends to the use of an extract of Pleurotus as set forth above, for the preparation of a pharmaceutical composition for the prevention and treatment of hypertension.

The invention also extends to a method of preparing an extract of Pleurotus comprising contacting a powder or pulp obtained from the mushroom or mushroom parts of the genus Pleurotus with one or more organic extraction solvents to remove an extract; and isolating the extract with antihypertensive activity. Most preferred solvents are disclosed herein.

The extract of this invention is shown to be effective in the prevention and treatment of hypertension in animal model systems. Additionally, since the extract is prepared from a natural, edible product, the potential for side effects is decreased.

As used herein and in the claims, the terms and phrases set out below have the meanings which follow.

“Active ingredient” means any extract or composition thereof capable of modifying or modulating the function of at least one given biological system.

“Antihypertensive effect” means a reduction in high blood pressure to more normal levels, i.e., systolic blood pressure remaining consistently lower than 140 mm Hg, or diastolic blood pressure remaining consistently lower than 90 mm Hg.

“Biocompatible” means generating no significant undesirable host response for the intended utility. Most preferably, biocompatible materials are non-toxic for the intended utility. Thus, for human utility, biocompatible is most preferably non-toxic to humans or human tissues.

“Carrier” means a suitable vehicle which is biocompatible and pharmaceutically acceptable, including for instance, one or more solid, semisolid or liquid diluents, excipients, adjuvants, flavours, or encapsulating substances which are suitable for administration.

The “extract EaxBu” or “EaxBu” is meant to refer to the ethyl acetate fraction obtained from Pleurotus eryngii (DC. et Fr.) Quél and which has antihypertensive activity.

“Extract” means a crude extract, purified extract, and purified composition obtained by solvent extraction and/or purification of the extract from a mushroom or parts thereof of the Pleurotus genus.

“Host” or “host animal” means humans or other invertebrates.

“Pharmaceutically- or therapeutically- or nutraceutically-effective” is used herein to denote any amount of a formulation of the extract which will exhibit an antihypertensive effect upon administration. The amount of extract administered will vary with the condition being treated, the stage of advancement of the condition, the age and type of host, and the type and concentration of the formulation being applied. Appropriate amounts in any given instance will be readily apparent to those skilled in the art or capable of determination by routine experimentation.

“Pharmaceutically- or therapeutically- or nutraceutically-acceptable” is used herein to denote a substance which does not significantly interfere with the effectiveness or the biological activity of the active ingredients (antihypertensive activity) and which has an acceptable toxic profile for the host to which it is administered.

“Mushroom or parts thereof” means either the whole mushroom, or any part of the mushroom.

“Pleurotus” is meant to refer to at least one mushroom of the Pleurotus genus including, but not limited to P. eryngii (DC. et Fr.) Quél., P. eryngii (DC. et Fr.) Quél. var. ferulae Lanzi, P. eryngii (DC. et Fr.) Quél. var nebrodensis Inzenga, P. citrinopileatus Sing., P. cornucopiae, P. cystidiosus, P. dryinus, P. eous, P. florida, P. griseas, P. olearius, P. ostreatoroseus Sing., P. ostreatus (Jacq. Ex Fr.) Quél., P. pulmonarius, P. sajor-caju (Fi.). Sing., P. salmomeo-stramineus L. Vasi., P. sapidus (Schulz) Sacc., P. serotinus (Schrad.) Fr., P. spodoleucus Fr., P. tuber-regium (Fi.) Sing., and P. ulmarius (Bull. Ex Fr.) Quél.

“Primary hypertension” or “essential hypertension” means hypertension of unknown etiology, but possibly of genetic or environmental origins (e.g., obesity, dietary sodium).

“Purified” means partially purified and/or completely purified. Thus, a “purified composition” may be either partially purified or completely purified.

“Secondary hypertension” means hypertension due to or associated with a variety of identifiable primary diseases such as renal disorders (e.g., polycystic renal disease, kidney inflammation), disorders of the central nervous system, endocrine diseases (e.g., adrenal gland tumors, Cushing's syndrome, hyperparathyroidism), and vascular diseases.

### BRIEF DESCRIPTION OF THE DRAWINGS

FIG. 1 is a flow diagram which illustrates the method to prepare the extract EaxBu from P. eryngii (DC. et Fr.) Quél.

FIG. 2 is a schematic illustration of the tissue-organ bath used for the determination of the effect of the extract EaxBu on isolated vascular tissue in vitro.

FIG. 3 is a graph showing the effect of the extract EaxBu (μg/ml) in varying concentrations upon pre-contracted rat aortic tissue.

FIG. 4 is a graph showing the effect of the extract EaxBu (mg/kg body weight) upon the mean arterial blood pressure (mm Hg) of normotensive Sprague-Dawley (SD) rats.

FIG. 5 is a graph showing the effect of the extract EaxBu (mg/kg body weight) upon the mean heartbeat (times/min) of normotensive SD rats.

FIG. 6 is a graph showing the effect of the extract EaxBu (mg/kg body weight) upon the mean blood pressure (mm Hg) of spontaneously hypertensive rats (SHR) compared to normotensive SD rats.

FIG. 7 is a graph showing the effects of the extract EaxBu (mg/kg body weight) upon the mean systolic blood pressure (mm Hg) of SHR compared to normotensive SD rats.

FIG. 8 is a graph showing the effect of the extract EaxBu (mg/kg body weight) upon the mean diastolic blood pressure (mm Hg) of SHR compared to normotensive SD rats.

FIG. 9 is a graph showing the effect of the extract EaxBu (mg/kg body weight) upon the mean heartbeat (time/min) of SHR over time.

### DETAILED DESCRIPTION OF THE INVENTION

i Preparation of Pleurotus Extract

The extract of the present invention is prepared from mushrooms in the genus of Pleurotus, most preferably from Pleurotus eryngii (DC. et Fr.) Quél. Other species of Pleurotus may be used, for instance P. eryngii (DC. et Fr.) Quél. var. ferulae Lanzi, P. eryngii (DC. et Fr.) Quél. var nebrodensis Inzenga, P. citrinopileatus Sing., P. cornucopiae, P. cystidiosus, P. dryinus, P. eous, P. florida, P. griseas, P. olearius, P. ostreatoroseus Sing., P. ostreatus (Jacq. Ex Fr.) Quél., P. pulmonarius, P. sajor-caju (Fi.). Sing., P. salmomeo-stramineus L. Vasi., P. sapidus (Schulz) Sacc., P. serotinus (Schrad.) Fr., P. spodoleucus Fr., P. tuber-regium (Fi.) Sing., and P. ulmarius (Bull. Ex Fr.) Quél. All Pleurotus species are edible and several are commercially cultivated, hence readily available.

Pleurotus eryngii (DC. et Fr.) Quél. is also known by the Chinese names of Xing Bao Gu, Yi Da Li Ce Er, Xing Xiang Bao Yu Gu, Xing Ren Bao Yu Gu, Xing Bao Rong, and Ci Qin Ce Er. The mushroom can be identified by its 2-11 cm diameter cap, which is grey, flat-ball shaped in a young mushroom and light yellow, circular or fan shaped in a mature mushroom. Cream colored gills are located under the surface of the cap, and the mycelium or cap meat is white with an apricot kernel flavor. The stalk is 2-8 cm high and 0.5-3 cm in diameter. P. eryngii (DC. et Fr.) Quél. is commercially available from reputable suppliers worldwide. The preparation of the extract “EaxBu” obtained from the whole P. eryngii (DC. et Fr.) Quél. mushroom (see Example 1), determination of its antihypertensive activity (see Examples 2 and 3), and formulations comprising the extract EaxBu are set forth herein.

In order to prepare extracts of Pleurotus, the mushroom or parts thereof may be provided as a powder (commercially available), or may be crushed and ground from a dry form of the mushroom or parts thereof to obtain a powder, or the mushrooms or parts thereof may be masticated to form a mushroom pulp or mash which can, if desired, be dried and ground. The powder or pulp can then be extracted with one or more organic extraction solvents. The solvent is then removed from the extract. The whole mushroom may be used or parts of the mushroom. Most preferably, the whole mushroom is used. If desired, the extract could be purified to yield a purified extract of one or more purified compositions using standard techniques such as column chromatography, fractional distillation, preparative TLC (thin layer chromatography), preparative HPLC (high performance liquid chromatography), CPC (Centrifugal Partition Chromatography) or other techniques known to those skilled in the art.

The extraction process of the present invention is desirably carried out using an organic or aqueous solvent or a mixture of organic extraction solvents. While ethyl acetate is the most preferred solvent used in the extraction process, other single solvents or solvent mixtures may be used. Preferred solvents have a dielectric constant (specific inductive capacity) of the solvent(s) in the range of ε=4.47 to 31.2 (see Bejing Medicinal College, 1980). Exemplary solvents include, alone or in admixture, acetone, benzyl acetate, butanol, butylacetate, chloroform, dichloromethane, ethanol, ether, ethyl formate, hexanol, hexanediol, isoamyl alcohol, isobutyl alcohol, methanol, pentanol, propanol, water and similar solvents. To assist in separating the extract of the present invention into the organic extraction solvent(s), other solvents with lower dielectric constants may be used for the discard fractions such as hexane, benzene, ether, petroleum ether, and chloroform.

ii Determination of Antihypertensive Activity

Once prepared, the extract EaxBu is evaluated to ensure antihypertensive activity by conducting one or more in vitro or in vivo pharmacological evaluations. In the present invention, such evaluations include, but are not limited to, an in vitro contractility assay and an in vivo blood pressure assay. Example 2 describes measurement of the effects of the extract EaxBu in vitro on isolated vascular tissue obtained from rats, while Example 3 determines the effects of the extract EaxBu in vivo following administration to normotensive and genetically hypertensive rats. For the present invention, any pharmacological evaluations are suitable, provided that they are focused upon indication of antihypertensive activity in either the extract or a representative sample from a batch of the extract in the event of large scale manufacturing.

iii Formulations, Formulating, Dosages and Treatment

a. Powders—Powders of the extract may be used in that form directly as a loose powder or encapsulated powder. Alternatively, powders may be formulated into capsules, caplets, tablets and similar dosage forms. Further, powders may be formulated within liquid pervious membranes such as filters, meshes and the like, such as a tea bag-type infuser, for generating liquids containing the dissolved extract.

b. Liquids—The powder form of the extract may be incorporated into liquids, formulated as solutions, dispersions or suspensions by dissolving the extract, for example as a drink, tincture, or drop. The extract may be administered alone, or with a carrier such as saline solution, an alcohol or water. An effective daily amount of the extract will vary with the subject, but will be less than is toxic while still providing a therapeutic effect. Solutions and formulations of the extract may lose some activity with aging and are thus either prepared in stable forms, or preferably prepared fresh for administration, for example in multicomponent kit form so as to avoid aging and to maximize the therapeutic effectiveness of the extract. Suitable kits or containers are well known for maintaining the phases of formulations separate until the time of use. A kit containing the extract in powder form may provide a sterile carrier such as water (and other ingredients) in a separate container in dosage specific amounts. The extract may be provided in a “tea bag”-type infuser or pouch, for generating liquid formulations at the time of use.

c. Sterilization, purification and/or microbiological assays—It will be understood that extracts and pharmaceutical compositions in the forms described above should be appropriately sterilized, purified and/or tested for microbiological parameters to ensure safety of administration. Extracts and pharmaceutical compositions should be sealed in appropriate packaging or containers which for example, limit moisture (as in the case of powders or encapsulated powders) which could impair the antihypertensive activity of the extract.

Typically, the extract will be formulated in one or more of the forms set out above. The extract can be prepared alone or as an active ingredient in pharmaceutical compositions including non-toxic, pharmaceutically acceptable carriers, diluents and excipients, as are well known, see for example Merck Index, Merck & Co., Rahway, N.J.; and Gilman et al., (eds) (1996) Goodman and Gilman's: The Pharmacological Bases of Therapeutics, 8^{th} Ed., Pergamon Press. For standard dosages of conventional pharmacological agents, see, e.g., Physicians Desk Reference (1997 Edition); and U.S. Pharmacopeia National Formulary (1995) United States Pharmacopeial Convention Inc., Rockville, Md. Compositions may also include flavors, colorings, coatings, etc. All agents must be non-toxic and physiologically acceptable for the intended purpose, and must not substantially interfere with the activity of the extract so as to deleteriously affect the antihypertensive effect. Ingredients are thus only included in therapeutically acceptable amounts.

The dosage of the extract depends upon many factors that are well known to those skilled in the art, for example, the particular form of the extract; the age, weight and clinical condition of the recipient patient; the concurrent therapeutic treatments; and the experience and judgement of the clinician or practitioner administering the therapy. A therapeutically effective amount of the extract provides either subjective relief of symptoms or an objectively identifiable improvement as noted by the clinician or other qualified observer. The extract may be administered orally, intraperitoneally, or intravenously at a dosage range and frequency (e.g., at least once daily) such that the level of active extract is maintained in the body. The dosage range varies with the route of administration, and the form and potency of the extract; for example, one dose of the extract in a capsule taken orally may contain for example 100-800 mg of the extract. The extract is preferably administered in spaced dosages throughout the day to maintain the level of active extract in the body. A dosage range between about 0.002-50 g is suggested (see Chen Qi, 1994). Preferably, the dosage range is 0.5-35 g, more preferably 2-25 g, and most preferably 9-15 g. Hypertension may thus be prevented or treated, and cardiovascular health enhanced by administering a therapeutically effective solution of the extract or pharmaceutical composition containing the extract.

Abbreviations and nomenclature employed herein are standard in the art and are commonly used in scientific publications such as those cited herein.

The invention is further illustrated by the following non-limiting examples.

### EXAMPLE 1

Preparation of the Ethyl Acetate Fraction of Pleurotus eryngii (DC. et Fr.) Quél Extract

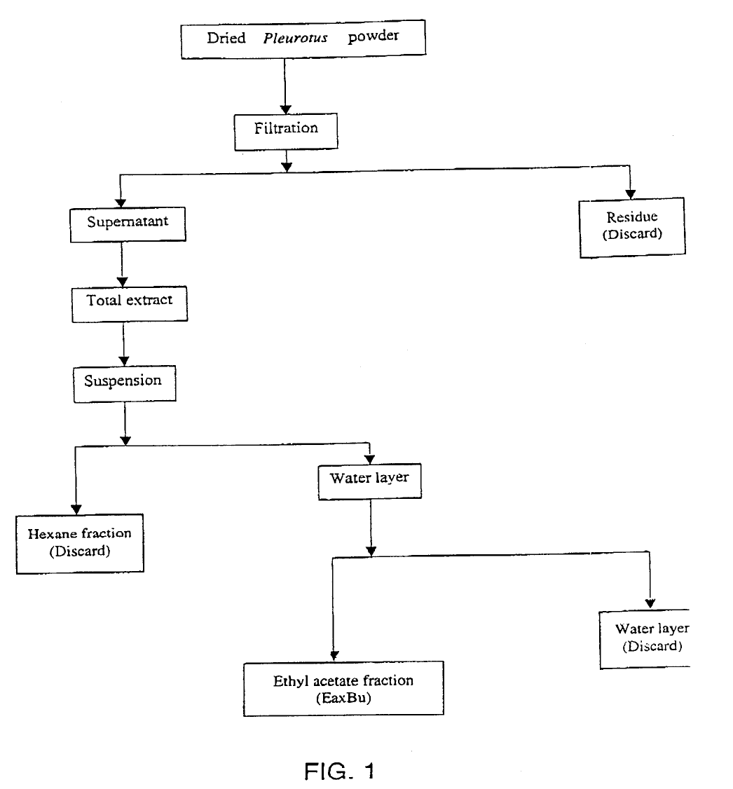

The method to prepare the ethyl acetate fraction of P. eryngii extract “EaxBu” is illustrated in FIG. 1. The first stage of the method involves obtaining a “total extract,” namely an extract from a fresh, whole P. eryngii (DC. et Fr.) Quél mushroom. If not already in the form of a powder, the P. eryngii (DC. et Fr.) Quél mushroom is first dried at room temperature for two weeks, and then dried in an oven at 35° C. for 48 hrs. The dry mushroom is ground into a powder using any commercially available blender. For this particular preparation, the Champ HP3™ blender is used at its highest setting (Model ES-3, K-TEC, Orem, Utah, USA). The powder is then soaked in 100% methanol at a ratio of 1 mg powder/5 ml methanol at room temperature for 24 hrs. A supernatant and precipitate are thus obtained. The supernatant is then filtered through filter paper (Grade 202, size 18.5 cm, Whatman Inc., Clifton, USA). The precipitate is saved for the next methanol soaping cycle. This step is repeated three to five times until the filtrate is clear.

The collected filtrate is then concentrated by evaporation to remove the methanol and to obtain the dry, “total extract” (representing the extract obtained from the whole P. eryngii (DC. et Fr.) Quél mushroom). The filtrate is evaporated at 45° C. using a standard evaporator (Buchi Rotavapor R-2000, BÜCHI Labortechnik AG, Flawil, Switzerland), and the obtained total extract is collected from the bottom of the evaporator flask. The yield of the dry, total extract prepared from the initial raw P. eryngii (DC. et Fr.) Quél powder is approximately 28.04% (by dry weight).

The second stage of the method involves diluting the dry, total extract with double distilled water at a ratio of 1 mg extract/10 ml water to form a suspension which is placed into a separating funnel. 100% hexane is added at a ratio of 10 ml hexane/10 ml suspension. The suspension/hexane mixture is stirred and left to settle until two layers have separated, with the hexane layer on the top and the water layer on the bottom.

The water layer on the bottom is released from the separating funnel into another container, and is re-extracted with hexane as described above at least five to six times until the hexane layer is clear. The collected water layers are saved to extract the ethyl acetate fraction.

The hexane layer is evaporated to dryness at 35° C. using a standard evaporator (Buchi Rotavapor R-2000, BÜCHI Labortechnik AG, Flawil, Switzerland), and the extract is collected from the bottom of the evaporator flask. The yield of this hexane fraction is 10.63% (by dry weight of the total extract).

In the third stage of the method, the extract EaxBu is obtained by separation with ethyl acetate. The collected water layers are placed into a separating funnel. 100% ethyl acetate is added at a ratio of 10 ml ethyl acetate/10 ml water layer. The mixture is stirred and left to settle until two layers have separated, with the ethyl acetate layer on the top and the water layer on the bottom.

The water layer on the bottom is released from the separating funnel into another container, and is re-extracted with ethyl acetate as described above at least five to six times until the ethyl acetate layer is clear.

The ethyl acetate layer is evaporated to dryness at 35° C. using a standard evaporator (Buchi Rotavapor R-2000, BÜCHI Labortechnik AG, Flawil, Switzerland), and the extract “EaxBu” is collected from the bottom of the evaporator flask. The yield of the extract EaxBu from total extract is approximately 5.28% (by dry weight). The yield of the extract EaxBu from the raw P. eryngii (DC. et Fr.) Quél powder is thus approximately 28.04%×5.28%=1.48%.

### EXAMPLE 2

Determination of the Antihypertensive Effect of the Extract EaxBu on Isolated Vascular Tissues in Vitro

One mechanism which contributes to lowering of high blood pressure involves vasodilation of the blood vessels. The ability of the extract EaxBu to induce vasodilation of isolated vascular tissues was examined in vitro. Male, eight to twelve week old Sprague-Dawley rats (278.86±6.85 g) were used in this study. Animals were housed in an animal care facility at the College of Medicine, University of Saskatchewan. The study was approved by the University Committee on Animal Care and Supply of the University of Saskatchewan.

Each animal was anaesthetized intraperitoneally with sodium pentobarbital (50 mg/kg). The thoracic cavity was incised, and the thoracic section of the aorta was gently dissected and placed into a Petri dish containing ice-cold Krebs' solution. Fat and connective tissues were removed from the aorta, and the aorta was then cut into six small rings (approximately 2 mm in width).

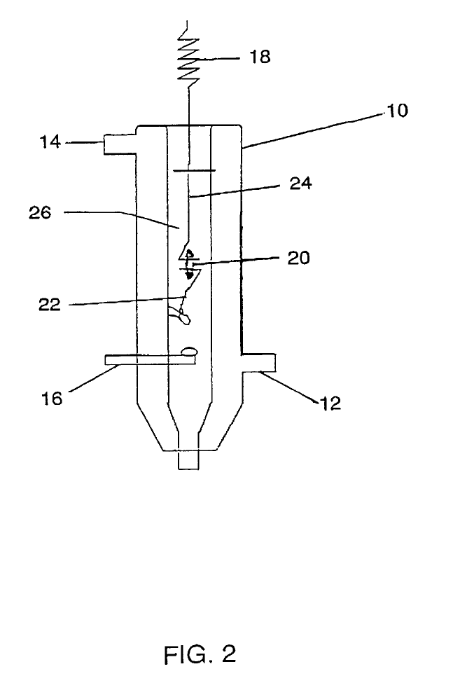

FIG. 2 is a schematic illustration of the tissue-organ bath 10 used in this study. Each of the six aortic rings was used in one tissue-organ bath 10; thus, six baths in total were used. Any standard tissue-organ bath can be used, for example those available from Grass-Telefactor (West Warwick, R.I., USA). The tissue-organ bath 10 typically has an inlet 12 and outlet 14 for water, and an inlet 16 for bubbling 95% oxygen, 5% carbon dioxide. A force displacement transducer 18 (Model FT03, Grass-Telefactor, West Warwick, R.I., USA) was used to measure the force development of the aortic ring 20. Data were recorded and analyzed using a Biopac System including the MP100WS acquisition units, transducer connector interface or amplifiers (TCI100), AcqKnowledge software (ACK100W, version 3.01) (all Biopac Systems, Inc., Santa Barbara, Calif., USA) and a standard IBM computer (not shown in FIG. 2).

The aortic ring 20 was secured in the tissue-organ bath 10 with a lower 22 and an upper 24 tungsten wires, with one end immobilized at lower wire 22 and the other tied by upper wire 24 to the force displacement transducer 18. The tissue-organ bath 10 was filled with 10 ml Krebs' bicarbonate solution 26 (115 mM NaCl, 5.4 mM KCl, 2.5 mM CaCl₂, 1.2 mM MgSO₄, 1.2 mM KH₂PO₄, 25.0 mM NaHCO₃, and 11 mM D-glucose) bubbled with 95% O₂ and 5% CO₂. The temperature was maintained at 37° C.

The aortic ring 20 was mechanically stretched to a basal tension of approximately 2.0 g and equilibrated for 1 hour prior to addition of any test agents. The responsiveness of the aortic ring 20 was initially tested by adding a sub-maximal concentration of phenylephrine (0.3 M) to induce contraction. After the plateau phase of contraction had been reached, the cumulative concentration-response relationship of the extract EaxBu was tested. The concentrations of the extract EaxBu were 0.2 μg/ml, 0.6 μg/ml, 2 μg/ml, 6 μg/ml, 20 μg/ml, 60 μg/ml, and 200 μg/ml. Only undamaged, responsive tissue was used in the experiment, which was confirmed by relaxation induced by 1 M acetylcholine at the end of the experiment.

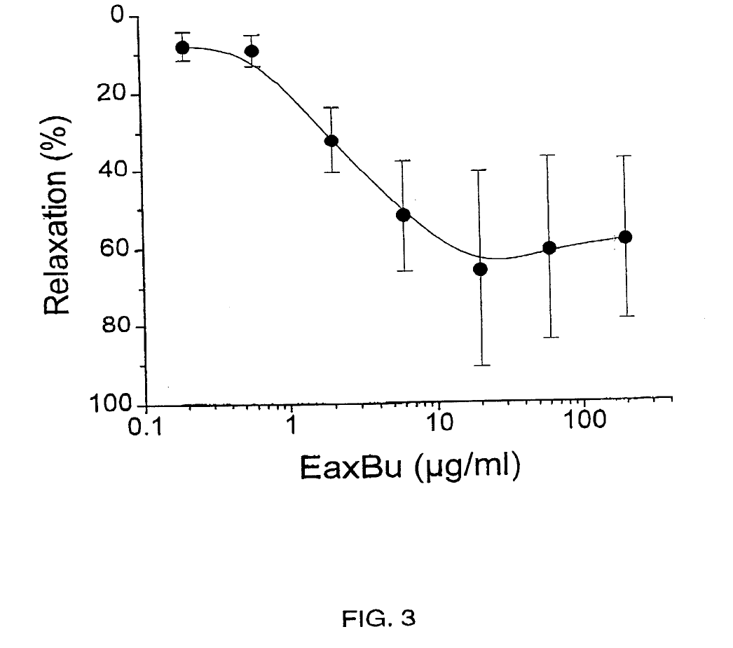

FIG. 3 is a graph showing the effect of the extract EaxBu in varying concentrations (expressed in logarithmic scale) upon the pre-contracted aortic tissue. The extract EaxBu relaxed aortic tissue in a concentration-dependent manner. Relaxation was initiated at a concentration beyond 0.6 μg/ml. The EC50 or concentration which provoked relaxation half way between the baseline and the maximum relaxation was 2.2±0.4 μg/ml. The maximal relaxation was 66.1±25.1% induced by 20 μg/ml of the extract EaxBu (n=6). This ability of the extract EaxBu to induce vasodilation of isolated vascular tissues in vitro demonstrates its efficacy in lowering blood pressure, hence preventing and treating hypertension.

### EXAMPLE 3

In Vivo Determination of the Antihypertensive Effect of the Extract EaxBu in a Rodent Model

The ability of the extract EaxBu to lower blood pressure was examined in vivo in a rodent model. Four twelve-week old male Sprague-Dawley rats (SD) and five twelve-week old male spontaneously hypertensive rats (SHR) were used in this study. The control group consisted of the SD rats, which are normotensive or have normal blood pressure. The test group consisted of the SHR, which innately have high blood pressure and are thus considered as a model of primary hypertension. SHR are generally used to determine the blood pressure lowering effects of antihypertensive compounds. Animals were housed in an appropriate animal care facility at the College of Medicine, University of Saskatchewan. The study was approved by the University Committee on Animal Care and Supply of the University of Saskatchewan.

Each animal was anaesthetised intraperitoneally with sodium pentobarbital (50 mg/kg). The animal was kept on a heating pad to maintain its body temperature at 37° C. throughout the experiment. One catheter was inserted into the left femoral artery, and connected to a pressure transducer to measure arterial blood pressure (Model AH 51-4844, Harvard Apparatus, Inc., Holliston, Mass., USA). A second catheter was inserted into the left femoral vein for intravenous injection of the extract EaxBu.

The animal's blood pressure was allowed to equilibrate for one hour before the first dose of the extract EaxBu was administered by intravenous injection. The doses were 0.35, 0.70, 1.75, 3.50, 14 and 28 mg/kg body weight. A 10 minute interval was maintained between injections. The instant mean blood pressure, mean systolic blood pressure, mean diastolic blood pressure, and mean heart rate values at 5 and 10 min after each injection were recorded and analyzed using a Biopac System including the MP100WS acquisition units, transducer connector interface or amplifiers (TCI100), AcqKnowledge software (ACK100W, version 3.01) (all Biopac Systems, Inc., Santa Barbara, Calif., USA) and a standard IBM computer.

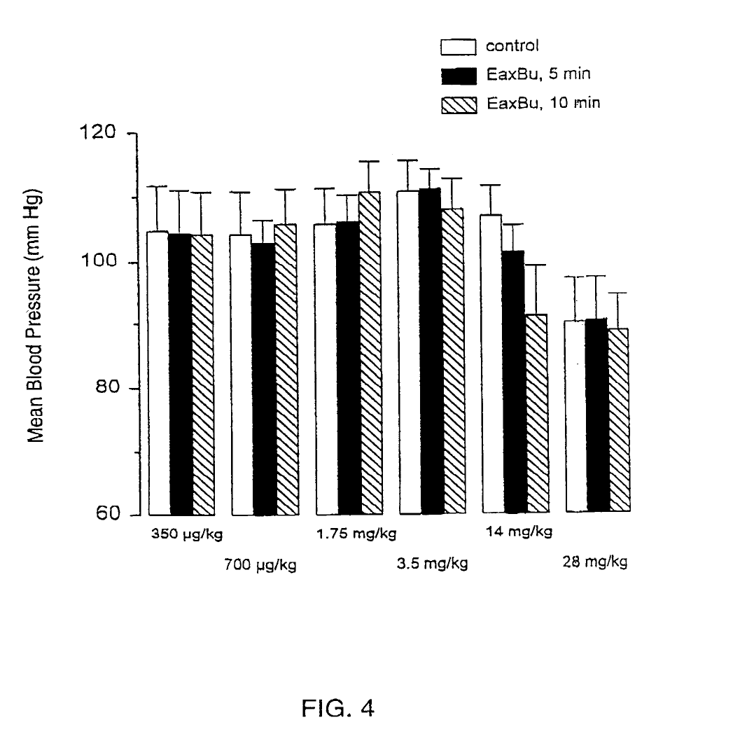

FIG. 4 is a graph showing the effect of the extract EaxBu (mg/kg body weight) upon the mean arterial blood pressure (mm Hg) of the control SD rats. The EaxBu extract had no significant effect, in that the mean blood pressure was virtually unchanged following the first 4 doses (up to 3.5 mg/kg body weight). The blood pressure started to decrease at a concentration of 14 mg/kg and decreased about 15 mmHg at a concentration of 28 mg/kg body weight. However, these changes were statistically non-significant (n=3).

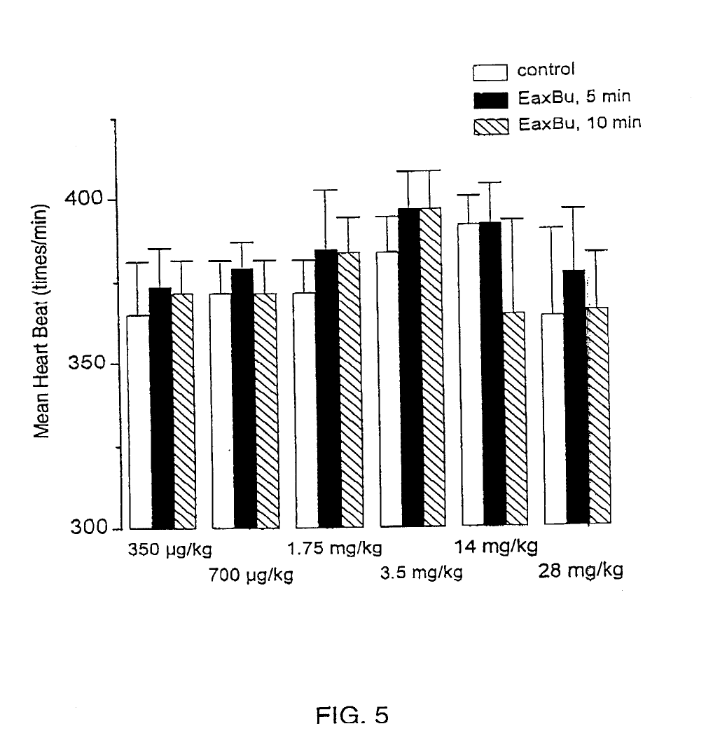

FIG. 5 is a graph showing the effect of the extract EaxBu (mg/kg body weight) upon the mean heartbeat (times/min) of the control SD rats. The simultaneous heart rate recording showed no obvious change in heart rate during administration of the extract EaxBu.

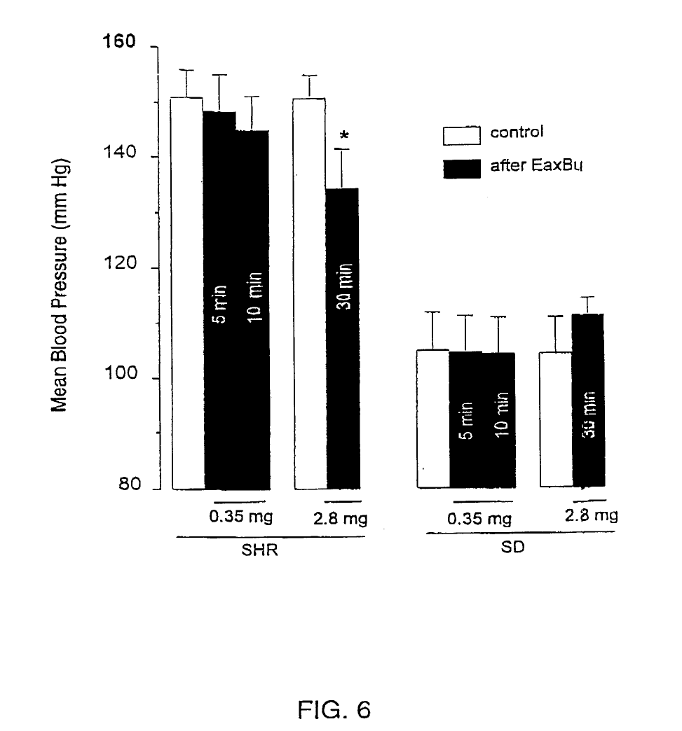

FIG. 6 is a graph showing the effect of the extract EaxBu (mg/kg body weight) upon the mean arterial blood pressure (mm Hg) of SHR compared to the control SD rats. In SHR, the extract EaxBu significantly decreased the mean arterial blood pressure 30 minutes after the injection of 2.8 mg/kg body weight (p<0.05 vs. control, n=5). This lowered blood pressure was maintained for approximately 45-60 minutes.

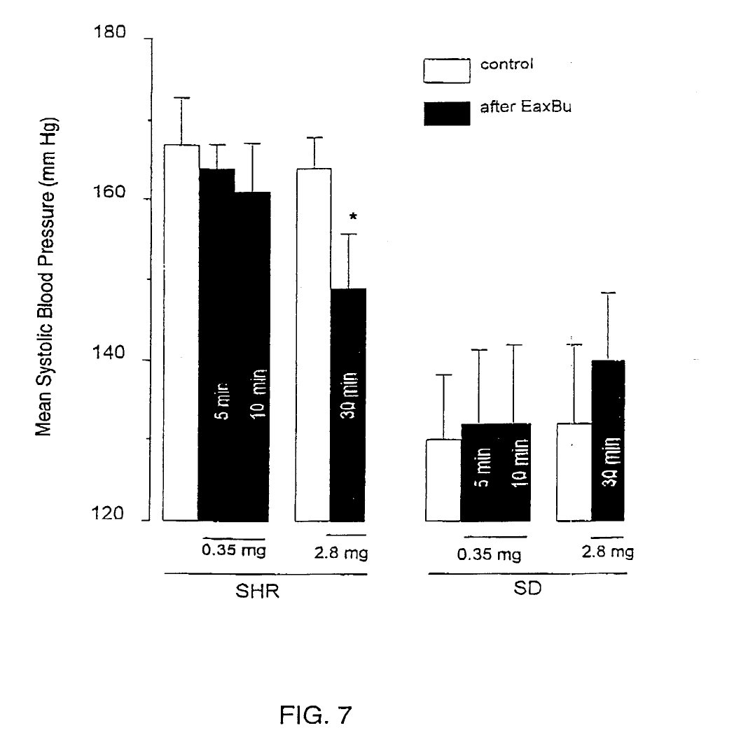

FIG. 7 is a graph showing the effects of the extract EaxBu (mg/kg body weight) upon the mean systolic blood pressure (mm Hg) of SHR compared to the control SD rats. The mean systolic blood pressure significantly decreased from 164±3.9 mm Hg to 149±6.8 mm Hg (p<0.05 vs. control, n=5) 30 minutes following administration of the extract EaxBu (2.8 mg/kg body weight).

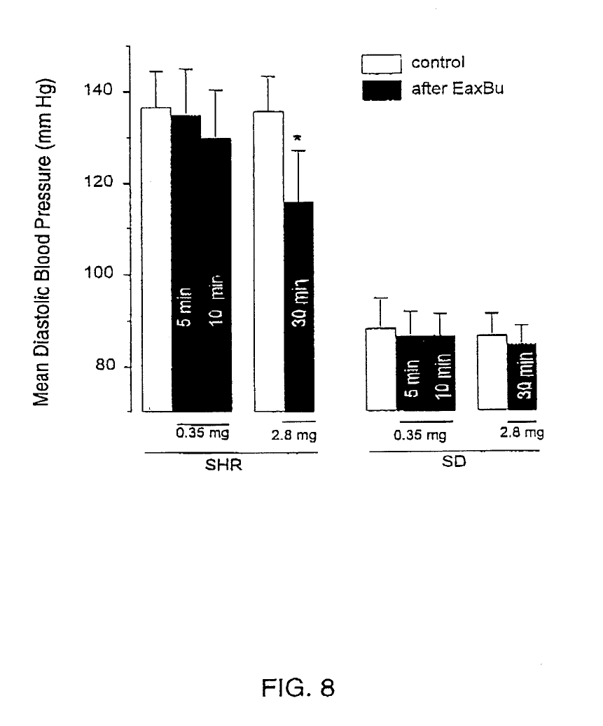

FIG. 8 is a graph showing the effect of the extract EaxBu (mg/kg body weight) upon the mean diastolic blood pressure (mm Hg) of SHR compared to the control SD rats. The mean diastolic blood pressure significantly decreased from 136±7.1 mm Hg to 116±11.3 mm Hg (p<0.05 vs. control, n=5) 30 minutes following administration of the extract EaxBu (2.8 mg/kg).

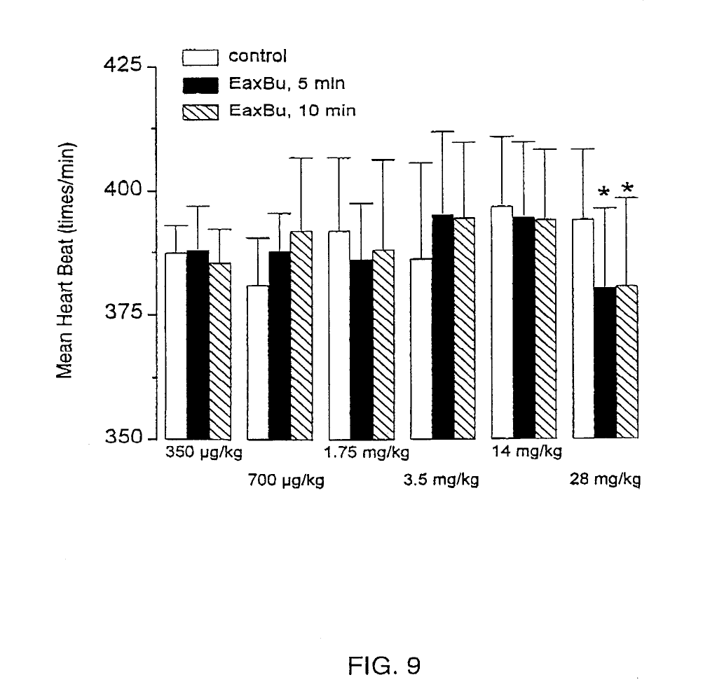

FIG. 9 is a graph showing the effect of the extract EaxBu (mg/kg body weight) upon the mean heartbeat (times/min) of SHR over time. Similar to the result obtained from the control SD rats, heart rate in SHR was not significantly affected by the extract EaxBu except at a high concentration of 28 mg/kg body weight (p<0.05 vs. control, n=5).

As demonstrated in Example 2, the potent ability of the extract EaxBu to induce vasodilation of isolated vascular tissues in vitro demonstrates its efficacy in lowering blood pressure, hence preventing and treating hypertension. Further, as demonstrated in the in vivo rodent studies in Example 3, the extract EaxBu effectively lowers the high blood pressure of genetically hypertensive rats, but does not affect the heart function or the normal blood pressure of normotensive control rats. The extract EaxBu thus demonstrates twofold activity, namely as a selective antihypertensive pharmaceutical drug to prevent and treat hypertension, and as an agent to enhance the cardiovascular health of the general population.

### REFERENCES

Beijing Medicinal College. 1980. Component Chemistry of Traditional Chinese Medicine. People's Hygiene Press. Beijing, China, pp. 17.

Chen, Qi. 1994. Methodology of Pharmacology Study for Traditional Chinese Medicine. People' Hygiene Press. Beijing, China, pp. 1103-5.

Gilman et al, (eds) (1996) Goodman and Gilman's: The Pharmacological Bases of Therapeutics, 8^{th} Ed., Pergamon Press.

Merck Index. Merck & Co., Rahway, N.J.

Physicians Desk Reference (1997 Edition)

U.S. Pharmacopeia National Formulary (1995) United States Pharmacopeial Convention Inc., Rockville, Md.

### Patent Literature

Akio, F. and Toshio, H. Food for sustaining normal blood pressure. Japanese Patent Application No. 61-109156, published Nov. 18, 1987.

Chihiro, S. Mushroom protein for food and beverage effective in preventing and treating hypertension and hyperlipemia and having antitumor action, mushroom protein for food and beverage effective in preventing and treating obesity and having antitumor action and method for extracting these proteins. Japanese Patent Application No. 07-120675, published Nov. 5, 1996.

Fumiharu, E. and Yasuo, W. Pleurotus eryngii strain, method for producing the same and hypertension therapeutic agent using the same. Japanese Patent Application No. P 2000-377553, published Jun. 25, 2002.

Kazuhide, A. Diabetes/hypertension/lever function improver comprising Panax notoginseng, fruit body of Ganoderma lucidum and Agaricus blazei murill as main components and its production. Japanese Patent Application No. 10-323664, published May 23, 2000.

Liu, X. and Chung, C. Germination activated Ganoderma lucidum spores and method for producing the same. U.S. Pat. No. 6,468,542, issued Oct. 22, 2002.

Masaru, O. And Imao, T. Food and medicine for prevention and remedy of hypertension, hyperlipemia and obesity. Japanese Patent Application No. 01-256515, published May 20,1991.

Satoshi, Y., Toshikazu, N., Satoshi, W., Mayumi, T., Michio, F. and Yoshikazu, S. Method for producing functional food by using Grifola frondosa. Japanese Patent Application No. 2000-156548, issued May 26, 2000.

Shigeru, Y., Yoshihiro, U. and Akio, F. Health food. Japanese Patent Application No. 57-216350, published Jun. 19, 1984.

Shoichi, I., Makio, K., Haruo, K. and Chieko, H. Physiologically active substance obtained from Basidiomycetes. Japanese Patent Application No. 05-270240, published May 16, 1995.

Takeshi, T., Shigeru, Y., Michitoku, K., Tadahito, T., Masaya, T., Masakatsu, M. and Norimasa, K. Medical composition of Ganoderma lucidum component. Japanese Patent Application No. 55-187752, published Jul. 13, 1982.

All publications mentioned in this specification are indicative of the level of skill in the art to which this invention pertains. To the extent they are consistent herewith, all publications mentioned in this specification are herein incorporated by reference to the same extent as if each individual publication was specifically and individually indicated to be incorporated by reference. No admission is made that any cited reference constitutes prior art.

Although the foregoing invention has been described in some detail by way of illustration and example, for purposes of clarity and understanding it will be understood that certain changes and modifications may be made without departing from the scope or spirit of the invention as defined by the following claims.

We claim:

1. An extract of Pleurotus obtained by solvent extraction from the whole mushroom or part thereof of the genus Pleurotus.

2. The extract of claim 1, wherein the mushroom is selected from the group consisting of P. eryngii (DC. et Fr.) Quél., P. eryngii (DC. et Fr.) Quél. var. ferulae Lanzi, P. eryngii (DC. et Fr.) Quél. var nebrodensis Inzenga, P. citrinopileatus Sing., P. cornucopiae, P. cystidiosus, P. dryinus, P. eous, P. florida, P. griseas, P. olearius, P. ostreatoroseus Sing., P ostreatus (Jacq. Ex Fr.) Quél., P. pulmonarius, P. sajor-caju (Fi.). Sing., P. salmomeo-stramineus L. Vasi., P. sapidus (Schulz) Sacc., P. serotinus (Schrad.) Fr., P. spodoleucus Fr., P. tuber-regium (Fi.) Sing., and P. ulmarius (Bull. Ex Fr.) Quél.

3. The extract of claim 2, wherein the mushroom is P. eryngii (DC. Fr.) Quél.

4. The extract of claim 3, wherein the extract is prepared from the whole mushroom.

5. The extract of claim 4, wherein the extract is obtained from an organic extraction solvent which includes ethyl acetate.

6. A pharmaceutical composition comprising an extract of Pleurotus obtained by solvent extraction from the whole mushroom or parts thereof of the genus Pleurotus, in admixture with one or more pharmaceutically acceptable carriers.

7. The pharmaceutical composition of claim 6, wherein the mushroom is selected from the group consisting of P. eryngii (DC. et Fr.) Quél., P eryngii (DC. et Fr.) Quél. var. ferulae Lanzi, P. eryngii (DC. et Fr.) Quél. var nebrodensis Inzenga, P. citrinopileatus Sing., P. cornucopiae, P. cystidiosus, P. dryinus, P. eous, P. florida, P. griseas, P. olearius, P. ostreatoroseus Sing., P. ostreatus (Jacq. Ex Fr.) Quél., P. pulmonarius, P. sajor-caju (Fi.). Sing., P. salmomeo-stramineus L. Vasi., P. sapidus (Schulz) Sacc., P. serotinus (Schrad.) Fr., P. spodoleucus Fr., P. tuber-regium (Fi.) Sing., and P. ulmarius (Bull. Ex Fr.) Quél.

8. The pharmaceutical composition of claim 7, wherein the mushroom is P. eryngii (DC. Fr.) Quél.

9. The pharmaceutical composition of claim 7, wherein the extract is prepared from the whole mushroom.

10. The pharmaceutical composition of claim 9, wherein the extract is obtained from an organic extraction solvent which includes ethyl acetate.

11. The pharmaceutical composition of claim 10, wherein the extract is in a dosage form to deliver 0.002 to 50 g of the extract on a daily basis.

12. The pharmaceutical composition of claim 11, wherein the extract is in a dosage form to deliver 0.5-35 g of the extract on a daily basis.

13. The pharmaceutical composition of claim 11, wherein the extract is in a dosage form to deliver 2-25 g of the extract on a daily basis.

14. The pharmaceutical composition of claim 11, wherein the extract is in a dosage form to deliver 9-15 g of the extract on a daily basis.

15. A method of preventing and treating hypertension comprising administering a therapeutically effective amount of an extract of Pleurotus obtained by solvent extraction from the whole mushroom or parts thereof of the genus Pleurotus to a host animal.

16. The method of claim 15, wherein the mushroom is selected from the group consisting of P. eryngii (DC. et Fr.) Quél., P. eryngii (DC. et Fr.) Quél. var. ferulae Lanzi, P eryngii (DC. et Fr.) Quél. var nebrodensis Inzenga, P. citrinopileatus Sing., P. cornucopiae, P. cystidiosus, P. dryinus, P. eous, P. florida, P. griseas, P. olearius, P. ostreatoroseus Sing., P. ostreatus (Jacq. Ex Fr.) Quél., P. pulmonarius, P. sajor-caju (Fi.). Sing., P. salmomeo-stramineus L. Vasi., P. sapidus (Schulz) Sacc., P. serotinus (Schrad.) Fr., P. spodoleucus Fr., P. tuber-regium (Fi.) Sing., and P. ulmarius (Bull. Ex Fr.) Quél.

17. The method of claim 16, wherein the mushroom is P. eryngii (DC. Fr.) Quél.

18. The method of claim 16, wherein the extract is prepared from the whole mushroom.

19. The method of claim 18, wherein the extract is obtained from an organic extraction solvent which includes ethyl acetate.

20. The method of claim 19, wherein the extract is in a dosage form to deliver 0.002 to 50 g of the extract on a daily basis.

21. The method of claim 20, wherein the extract is in a dosage form to deliver 0.5-35 g of the extract on a daily basis.

22. The method of claim 20, wherein the extract is in a dosage form to deliver 2-25 g of the extract on a daily basis.

23. The method of claim 20, wherein the extract is in a dosage form to deliver 9-15 g of the extract on a daily basis.

24. A method of preparing an extract of Pleurotus, comprising:
contacting a powder or pulp obtained from the mushroom or mushroom parts of the genus Pleurotus with one or more organic extraction solvents to remove an extract; and
isolating the extract with antihypertensive activity.

25. The method of claim 24, wherein the mushroom is selected from the group consisting of P. eryngii (DC. et Fr.) Quél., P. eryngii (DC. et Fr.) Quél. var. ferulae Lanzi, P. eryngii (DC. et Fr.) Quél. var nebrodensis Inzenga, P. citrinopileatus Sing., P. cornucopiae, P. cystidiosus, P. dryinus, P. eous, P. florida, P. griseas, P. olearius, P. ostreatoroseus Sing., P. ostreatus (Jacq. Ex Fr.) Quél., P. pulmonarius, P sajor-caju (Fi.). Sing., P. salmomeo-stramineus L. Vasi., P. sapidus (Schulz) Sacc., P. serotinus (Schrad.) Fr., P. spodoleucus Fr., P. tuber-regium (Fi.) Sing., and P. ulmarius (Bull. Ex Fr.) Quél.

26. The method of claim 25, wherein the mushroom is P. eryngii (DC. Fr.) Quél.

27. The method of claim 25, wherein the extract is prepared from the whole mushroom.

28. The method of claim 27, wherein the extract is obtained from an organic extraction solvent which includes ethyl acetate.

29. The method of claim 28, wherein the extract is prepared by:
contacting a powder or pulp derived from the mushroom with a first solvent which includes methanol, followed by filtration and solvent removal to obtain a total extract;
contacting the total extract with water and hexane and recovering a first water fraction; and
contacting the first water fraction with ethyl acetate, recovering a second water fraction and removing the ethyl acetate to obtain an extract.
